(19) United States
(12) Patent Application Publication
(10) Pub. No.: US 2016/0270868 A1
(43) Pub. Date: Sep. 22, 2016

(54) Thread Handling Tool

(71) Applicant: Cambridge Medical Robotics Limited, Cambridge (GB)

(72) Inventor: Steven James Randle, Woking (GB)

(73) Assignee: Cambridge Medical Robotics Limited

(21) Appl. No.: 15/075,940

(22) Filed: Mar. 21, 2016

(30) Foreign Application Priority Data
Mar. 20, 2015 (GB) 1504788.9

(51) Int. Cl. A61B 34/30; A61B 17/04; A61B 17/00

(52) U.S. Cl. CPC A61B 34/30; A61B 17/00234; A61B 17/0469

(57) ABSTRACT

A surgical instrument including an elongate shaft terminating in an end effector, the end effector including two pairs of opposable jaws, the pairs being spaced apart and independently operable to cause the jaws of that pair to grip or release an object located therebetween, wherein the jaws of each pair are movable to an open configuration in which the most distal point on each of those jaws is less distal than the mid-point of that pair of jaws when that pair of jaws is closed with its jaws in their most distally extended configuration.

### REFERENCE TO RELATED APPLICATION

This application claims priority to United Kingdom Patent Application No. 1504788.9, entitled “Thread Handling Tool,” filed Mar. 20, 2015, the entirety of which is hereby incorporated by reference herein.

### FIELD OF INVENTION

The present disclosure relates to a tool usable for handling thread, for example suturing thread.

### BACKGROUND

Laparoscopic surgery can be conducted manually or using surgical robots. One action that is commonly required as part of a laparoscopic procedure is to perform suturing, for example to close cuts or lesions in tissue that is being operated on. The tissue to be sutured may be on the surface of a patient's body, or it may be internal to the patient. When internal suturing is being carried out by laparoscopic techniques, the path to access the suturing site is normally very confined, meaning that there is limited mobility of surgical instruments at the site. This makes it difficult to perform suturing in laparoscopic surgery. When student surgeons are being taught to perform laparoscopic surgery the skills required for suturing often occupy a substantial part of the training time.

One technique that causes particular difficulty is passing an end of thread behind another length of thread. FIG. 1 illustrates this problem. FIG. 1 shows surgical instruments working within the body of a patient. A length of thread 1 is held in the jaw 2 of a first laparoscopic instrument 3. A second length of thread 4 terminates in a free end 5. Length 4 is held in the jaw 6 of a second laparoscopic instrument 7. Suppose it is required to pass length 4 behind length 1. (The word “behind” is used to denote that length 4 is to pass on the side of length 1 that is further from the entry points 8, 9 of the instruments 3, 7 into the patient's body). This action might be required as part of the process of tying a suture around tissue 10. To pass length 4 behind length 1, length 4 is gripped by jaw 6 while length 1 is held by jaw 2. Instrument 7 is moved so as to guide the free end 5 of length 4 behind length 1. This motion can only proceed up to the point when jaw 6 abuts length 1. At that point, jaw 6 is operated to release length 4. Jaw 6 can then be moved to the opposite side of length 4, where it is operated to grip length 4 again. Then jaw 6 can be used to draw further thread from length 4 behind length 1.

A difficulty with this procedure is that during the step when jaw 6 is moved to the opposite side of length 1, length 4 is freely floating. As a result, it is possible for length 4 to be displaced, for example due to blood flow in the region where the suturing is being conducted. This is time-consuming because the surgeon then needs to re-acquire a grip of thread length 4, and perhaps repeat the process of passing it behind length 1. This increases cost, increases fatigue of the surgeon, and may expose the patient to additional risk associated with a longer duration of operation.

One way to avoid the free end of the thread floating freely is to use a third instrument to grip the free end before jaw 6 is released. The disadvantage of this is that introducing a third instrument to the operating site involves additional disruption to the patient's tissues.

There is a need for an improved way of handling thread in performing laparoscopic procedures.

### SUMMARY

According to one aspect of the present disclosure there is provided a surgical instrument comprising an elongate shaft terminating in an end effector, the end effector comprising two pairs of opposable jaws, the pairs being spaced apart and independently operable to cause the jaws of that pair to grip or release an object located therebetween, wherein the jaws of each pair are movable to an open configuration in which the most distal point on each of those jaws is less distal than the mid-point of that pair of jaws when that pair of jaws is closed with its jaws in their most distally extended configuration.

The jaws of a first one of the pairs may be spaced from the jaws of the other one of the pairs.

The jaws of each pair may be rotatable relative to the remainder of the instrument in a common plane. The common planes of each pair of jaws may be mutually parallel.

For each pair of jaws, the pivot axis of one jaw of the pair may be parallel to the pivot axis of the other jaw of the pair. The pivot axes of both jaws of both pairs of jaws may be parallel.

The pivot axis of one jaw of each pair of jaws may be aligned with the pivot axis of the other jaw of that pair of jaws in the longitudinal direction of the shaft. The pivot axes of both jaws of both pairs of jaws may be aligned in the longitudinal direction of the shaft.

The pivot axes of both jaws of each pair of jaws may be transverse to the longitudinal axis of the shaft. The pivot axes of both jaws of both pairs of jaws may be transverse to the longitudinal axis of the shaft.

The jaws of each pair may be movable to an open configuration in which the most distal point on each of those jaws is less distal than the mid-point of the other pair of jaws when that other pair of jaws is closed with its jaws in their most distally extended configuration.

The surgical instrument may have at its distal end an interface to a surgical robot or a manually operable interface whereby the configuration of the jaws can be altered.

The instrument may be a laparoscopic instrument.

According to a second aspect of the present disclosure there is provided a method for manipulating a first elongate element to pass behind another elongate element by means of a surgical instrument as set out above, the method comprising the following steps in order: gripping the first elongate element with a first pair of the jaws; with the second pair of the jaws in an open configuration, causing the second elongate element to pass between the first elongate element and the second pair of the jaws whilst the first elongate element remains gripped by the first pair of the jaws; gripping the first elongate element with the second pair of the jaws whilst the first elongate element remains gripped by the first pair of the jaws; releasing the first elongate element with the first pair of the jaws whilst the first elongate element remains gripped by the second pair of the jaws; and with the first pair of the jaws in an open configuration, causing the second elongate element to pass between the first elongate element and the first pair of the jaws whilst the first elongate element remains gripped by the second pair of the jaws.

The method may comprise maintaining a grip of the first elongate element with at least one pair of the jaws throughout the said steps.

The method may comprise the step, subsequent to the steps set out above, of drawing a further length of the first elongate element behind the second elongate element.

Each elongate element may be a length of suturing thread. The lengths of suturing thread may be parts of the same run of suturing thread.

These embodiments are mentioned not to limit or define the limits of the present subject matter, but to provide examples to aid understanding thereof. Illustrative embodiments are discussed in the Detailed Description, and further description is provided there. Advantages offered by various embodiments may be further understood by examining this specification and/or by practicing one or more embodiments of the claimed subject matter.

Aspects of the present disclosure will now be described by way of example with reference to the accompanying drawings.

### BRIEF DESCRIPTION OF DRAWINGS

FIG. 1 illustrates a technique for passing one thread length behind another according to some aspects of the present disclosure.

FIG. 2 illustrates a surgical instrument having two gripping jaws according to some aspects of the present disclosure.

FIG. 3 is a partial cross-section on line A-A′ of FIG. 2 through the end effector of the instrument according to some aspects of the present disclosure.

FIG. 4 illustrates a stage in the process of handling a thread in a laparoscopic procedure using the instrument of FIG. 2 according to some aspects of the present disclosure.

FIG. 5 illustrates a side view of an instrument shown in FIG. 4 according to some aspects of the present disclosure.

### DETAILED DESCRIPTION

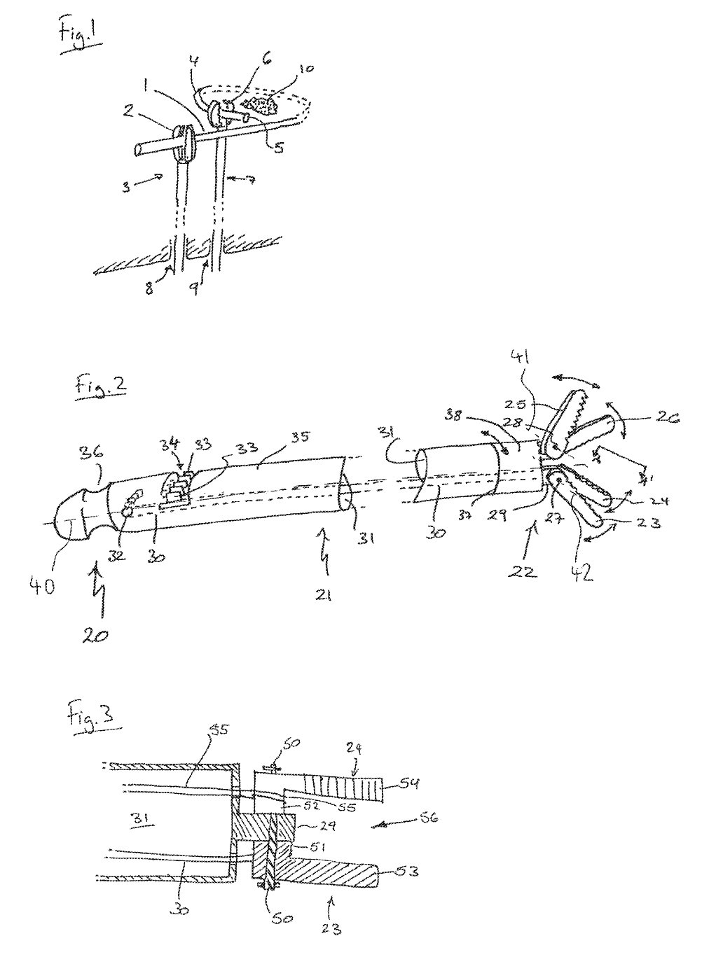

The instrument shown in FIG. 2 is capable of passing one thread (A) behind another thread (B) whilst not letting go of the former thread. It has two opposable pairs of jaws, the pairs being spaced apart from each other. With a leading pair of the jaws open and a trailing pair of the jaws gripping thread A, the instrument can be manipulated so that thread B passes between the leading pair of jaws and thread A, until the point where thread B is located in front of thread A and between one opposable pair of jaws and the other. Then the leading pair of jaws can be closed, and subsequently the trailing pair of jaws can opened. Next, the instrument can be manipulated so that thread B passes between the trailing jaws and thread A, freeing the instrument from thread B and leaving thread A behind thread B. Finally, since the instrument is free of thread B the instrument can be moved away from thread B, drawing more of thread A behind thread B.

FIG. 2 shows a surgical instrument. The instrument comprises a proximal end 20 for attachment to a surgical robot, an elongate body 21 and an end effector 22.

The end effector includes four jaws 23, 24, 25, 26. Jaws 23 and 25 are opposed: they can come together to grip an object. Jaw 23 pivots about an axis 42 at joint 27. Jaw 25 pivots about an axis 41 at joint 28. Jaws 24 and 26 are opposed: they can come together to grip an object. Jaw 24 pivots about axis 42 at joint 27. Jaw 26 pivots about axis 41 at joint 28. Pivot axis 41 may be parallel to pivot axis 42. Pivot axis 41 may be aligned with the pivot axis 42 in the direction of elongation of the instrument shaft 21. In other words, the pivot axis 41 and the pivot axis 42 may be aligned in the longitudinal direction of the shaft which is parallel to the longitudinal axis 40 of the shaft. Both the pivot axis 41 and the pivot axis 42 may be transverse to the longitudinal axis of the shaft. Jaws 23 and 24 can pivot about a common axis 42 at joint 27. Jaws 25 and 26 can pivot about a common axis 41 at joint 28. The axis 42 of joint 27 is parallel to the axis 41 of joint 28 but offset from it in a direction perpendicular to the direction of elongation of the instrument shaft 21. In other words, the axis 42 is parallel to but offset from axis 41 in a direction transverse to the longitudinal direction of the shaft.

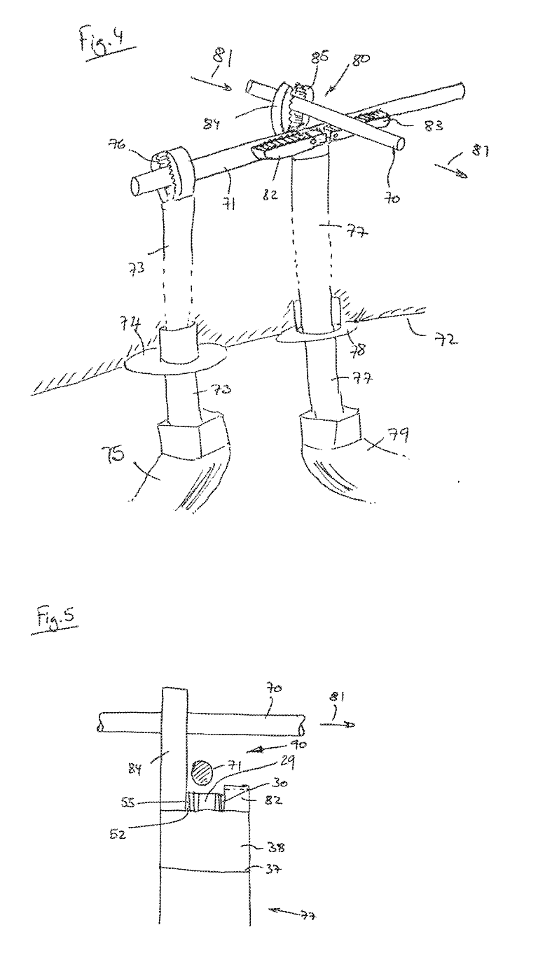

At each joint there is a pin (e.g. pin 50 in FIG. 3) borne by a joint block 29 of the instrument. The joint block is attached to the instrument shaft and carries the jaws. The respective jaw is threaded on to the pin and has a barrel or spool formation 51, 52, of circular exterior surface, which surrounds the pin. The barrel formation is rotationally fast with the gripping arm 53, 54 of the respective jaw. Closed loops of flexible filament 30, 55 wind tightly around respective ones of the barrel formations. For clarity only some of the loops of filament are shown in FIG. 2.

The loops of filament extend from the end effector of the instrument, through a channel 31 in the hollow interior of the body 21, and then around a respective pulley 32 which is borne by the proximal end of the instrument. Attached to each loop of filament is a respective flag 33. The flags are exposed through a window 34 in the outer wall 35 of the instrument body. The loops of filament can be tensioned by springs drawing the pulleys 32 in a proximal direction or by a turnbuckle located on the loop, for example at the respective flag 33.

The proximal end of the instrument could be equipped with any suitable structures to permit it to be attached to a certain type of robot. In this example the proximal end of the instrument is configured with a convex annular groove 36. This allows it to be clipped into place in a correspondingly shaped socket on the distal end of a surgical arm. The mating formation on the instrument could be of suitable shape to mate with any particular design of surgical robot. The arm of the surgical robot can have a series of linear actuators each of which is positioned to engage with a respective one of the flags when the instrument is mated to the arm. The linear actuators could, for example, be ball screws or hydraulic pistons. Once the instrument is attached to the arm in this way the linear actuators can be controlled to move any one of the flags along the axis of the instrument. This causes the loop of filament attached to that flag to move along its length, turning the barrel formation around which it is wound and causing the respective jaw to pivot around its joint. In this way the jaws can be controlled to pivot independently, allowing in operator to bring the opposing pairs of jaws into engagement or into a splayed configuration in which they are spread apart.

The end effector has a further joint 37 which allows a terminal part 38 of the instrument to rotate about an axis parallel to the direction of elongation of the instrument. Rotation about that joint is controlled by motion of one of the flags 33 to move a further loop of filament which acts on the terminal part 38.

The pairs of jaws that are mounted on a common axis are spaced apart. In the example of FIG. 3, they are spaced by the width of the block 29 and the barrels 51, 52. This means that it is possible for material, for example suture thread, to pass between jaws 23 and 24 and between jaws 25 and 26. This gap is shown at 56 in FIG. 3.

Each jaw may be provided with a serrated, roughened or soft surface on its face that can be brought into contact with its opposing jaw. This can help to improve its gripping qualities.

The instrument shown in FIGS. 2 and 3 can be used to pass a first suture thread behind a second suture thread without letting go of the first suture thread. FIG. 4 illustrates how this is done.

FIG. 4 shows a first length 70 of suture thread and a second length 71 of suture thread. The lengths of thread are located inside the body of a patient on whom surgery is being performed. The patient's skin is shown at 72.

Two surgical instruments are being used inside the patient. Instrument 73 passes into the patient's body through a port 74 at the patient's skin. The proximal end of instrument 73 is attached to the distal end of a first robot arm 75. Robot arm 75 can manipulate the location of the end effector 76 of instrument 73 within the body of the patient, and can operate the jaws of the end effector to open and close them as required. The other instrument 77 passes into the patient's body through a port 78 at the patient's skin. The proximal end of instrument 77 is attached to the distal end of a first robot arm 79. Robot arm 79 can manipulate the location of the end effector 80 of instrument 77 within the body of the patient, and can operate the jaws of the end effector to open and close them as required.

FIG. 4 shows instrument 77 in the act of passing thread length 70 behind thread length 71, with thread length 70 being passed in the direction shown by arrows 81. Instrument 77 is an instrument of the type shown in FIGS. 2 and 3, with two independently operable, spaced apart sets of gripping jaws. Instrument 77 has a first pair of opposed jaws 82, 83, which can be moved to grip an object between themselves, or moved mutually apart; and a second pair of opposed jaws 84, 85, which can be moved to grip an object between themselves, or moved mutually apart. Jaw pair 82, 83 is further advanced in the direction of motion of thread length 70 than jaw pair 84, 85 and so jaw pair 82, 83 will be referred to as the leading jaws. Jaw pair 84, 85 will be referred to as the trailing jaws.

An example of a sequence of events for passing thread length 70 behind thread length 71 using instrument 77 will now be described.
1. In a first stage of the process, the thread length 70 that is to be passed behind another thread length 71 is gripped in the trailing jaws 84, 85. Thread 70 is gripped in such a way that some of thread 70 is also located in the plane of motion of the leading jaws 82, 83, so that it can also be gripped by the leading jaws without movement between it and the trailing jaws.
2. If the leading jaws are not already open, they are opened to the extent that a gap is produced between the leading jaws and the thread 70 that is being gripped by the trailing jaws.
3. One or both of the instruments is/are manipulated by their respective arms so as to cause the length 71 of thread that is to pass in front of thread 70 to pass through the gap between the leading jaws and thread 70, to a position where:
   (a) the thread 71 that is to pass in front is located between (i) the thread 70 that is to pass behind and (ii) and the body of the instrument 77 holding the thread 70 that is to pass behind; and
   (b) the thread 71 that is to pass in front is located between the leading jaws 84, 85 and the trailing jaws 82, 83.
      This state is illustrated in FIG. 4, and in FIG. 5 which shows a side view of end effector 80. Parts not specifically discussed above in relation to FIG. 4 are numbered in FIG. 5 as in FIGS. 2 and 3. The gap between thread 70 and the leading jaws 82, 83 (jaw 83 not being visible in FIG. 5) is shown at 90.
4. The leading jaws 82, 83 are operated to grip the thread 70. At this point the trailing jaws are still gripping thread 70 so the thread is not freely floating.
5. The trailing jaws 84, 85 are operated to release their grip on thread 70, and are opened to the extent that a gap is produced between the trailing jaws and the thread 70 that is now being gripped by the leading jaws.
6. One or both of the instruments is/are manipulated by their respective arms so as to cause the front thread length 71 to in front to pass through the gap between the trailing jaws and the rear thread length 70.
7. Now the end effector 80 of instrument 77 is free from thread 71, and instrument 77 can be manipulated to draw further thread of length 70 behind thread 71.
   As indicated above, this process allows one thread to be drawn behind the other whilst both thread are continually being gripped. This can make the task of suturing considerably easier.

Thread lengths 70 and 71 could be parts of the same run of thread. Conveniently, thread length 70 terminates in a free end that is displaced from end effector 80 in direction 81 but is nevertheless close to end effector 80: for example within five times the width of end effector 80 in direction 81 from the end effector itself.

To allow the front-most thread to pass readily between the leading jaws and the rear-most thread, and to allow the rear-most thread to pass between the trailing jaws and the rear-most thread, it is preferred that each set of jaws can be opened to a substantial extent so that its most distal point is significantly proximal of the most distal end of the gripping set of jaws. For example, each set of jaws may be openable so that in at least one configuration of the jaw pairs that set of jaws is fully proximal of a point half-way along the length of the jaws of the other set. That configuration may be when the said other set is arranged to extend as distally as possible. Each jaw is capable of rotating by at least 70°, and in some examples can rotate by 80° or 90°, in an opening direction from its most distally extending position.

In an exemplary implementation, each set of jaws can be opened to a substantial extent such that its most distal point when open is significantly proximal of its most distal point when closed in a straight configuration of the jaws. For example, each set of jaws may be movable to at least one open configuration in which that set of jaws is fully proximal of a point half-way along the length of that same set of jaws when closed with its jaws in their most distally extended configuration.

In the examples above the instruments are adapted for attachment to surgical robot arms. The instruments could alternatively be for manual laparoscopic surgery. In that case the proximal end of the instruments can be provided with manually actuable grips, buttons, plungers or other means to allow a surgeon to operate both pairs of jaws separately. In the case of a surgical robot, the robot may be provided with a dedicated user interface control that swaps the gripping action from one pair of jaws to the other in such a way that a pair of jaws that was initially gripping is automatically not released until the other set of jaws is gripping.

The control linkage from the proximal end of the instrument to the distal end of the instrument could be provided by any suitable means, for example flexible control lines, rigid control rods, concentric hollow channels, or by pneumatic, hydraulic or electrical drive from the proximal end of the instrument to actuators in the distal end of the instrument.

Each jaw could be operable independently of the others. Alternatively, the instrument could be arranged so that the jaws of a pair move together either inwards, to grip an object, or apart. The mechanisms of both sets of jaws could be interlinked, so that as a controller is moved from a first position to a second position the jaws adopt, in sequence, the conditions (i) one jaw pair closed, other jaw pair open, (ii) both jaw pairs closed, (iii) the said one jaw pair open, the said other jaw pair closed. For example, in the case of a manual surgical tool the proximal end of the tool could be provided with a first hand-engaging part and a second hand-engaging part, one part being movable relative to the other to control the two pairs of jaws of the instrument and the handle being coupled to the control linkages of the instrument so that continuous travel of one hand-engaging part relative to the other brings about the three conditions listed above, in that sequence.

The instrument could be used for passing any suitable elongate object behind another. For example, each elongate object could independently be any of: suturing or other thread; a cannula, catheter, stent or other tube; a needle; a guide wire; an electrical wire; an optical fibre; or a tissue structure such as a blood vessel, tendon, ligament or nerve.

The instrument could be used for non-surgical purposes. For example, it could be used for knotting dental floss in a cosmetic procedure.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. Aspects of the present invention may consist of any such individual feature or combination of features. In view of the foregoing description, it will be evident to a person skilled in the art that various modifications may be made within the scope of the invention.

1. A surgical instrument comprising an elongate shaft terminating in an end effector, the end effector comprising two pairs of opposable jaws, the pairs being spaced apart and independently operable to cause the jaws of that pair to grip or release an object located therebetween, wherein the jaws of each pair are movable to an open configuration in which the most distal point on each of those jaws is less distal than the mid-point of that pair of jaws when that pair of jaws is closed with its jaws in their most distally extended configuration.

2. A surgical instrument as claimed in claim 1, wherein the jaws of a first one of the pairs are spaced from the jaws of the other one of the pairs.

3. A surgical instrument as claimed in claim 1, wherein the jaws of each pair are rotatable relative to the remainder of the instrument in a common plane.

4. A surgical instrument as claimed in claim 3, wherein the common planes of each pair of jaws are mutually parallel.

5. A surgical instrument as claimed in claim 1, wherein for each pair of jaws, the pivot axis of one jaw of the pair is parallel to the pivot axis of the other jaw of the pair.

6. A surgical instrument as claimed in claim 5, wherein the pivot axis of one jaw of the pair is aligned with the pivot axis of the other jaw of the pair in the longitudinal direction of the shaft.

7. A surgical instrument as claimed in claim 6, wherein the pivot axes of both jaws of the pair are transverse to the longitudinal axis of the shaft.

8. A surgical instrument as claimed in claim 5, wherein the pivot axes of both jaws of both pairs of jaws are parallel.

9. A surgical instrument as claimed in claim 6, wherein the pivot axes of both jaws of both pairs of jaws are aligned in the longitudinal direction of the shaft.

10. A surgical instrument as claimed in claim 7, wherein the pivot axes of both jaws of both pairs of jaws are transverse to the longitudinal axis of the shaft.

11. A surgical instrument as claimed in claim 1, wherein the jaws of each pair are movable to an open configuration in which the most distal point on each of those jaws is less distal than the mid-point of the other pair of jaws when that other pair of jaws is closed with its jaws in their most distally extended configuration.

12. A surgical instrument as claimed in claim 1, having at its distal end an interface to a surgical robot whereby the configuration of the jaws can be altered.

13. A surgical instrument as claimed in claim 1, having at its distal end a manually operable interface whereby the configuration of the jaws can be altered.

14. A surgical instrument as claimed in claim 1, the instrument being a laparoscopic instrument.
